(19) United States
(12) Patent Application Publication
(10) Pub. No.: US 2007/0219201 A1
(43) Pub. Date: Sep. 20, 2007

(54) COMBINATION OF ATOMOXETINE AND A 5HT1A RECEPTOR AGONIST FOR TREATING ADHD AND OTHER DISORDERS

(75) Inventors: Richard T. Carroll Jr., Toledo, OH (US); Lamia Sharmeen, Ann Arbor, MI (US)

Correspondence Address: WARNER-LAMBERT COMPANY, 2800 PLYMOUTH RD, ANN ARBOR, MI, 48105, US

(73) Assignee: WARNER-LAMBERT COMPANY LLC, Morris Plains, NJ (US)

(21) Appl. No.: 11/569,644

(22) PCT Filed: May 13, 2005
(86) PCT No.: PCT/IB05/01450

§ 371 Date: Nov. 27, 2006

Related U.S. Application Data
(60) Provisional application No. 60/574,751, filed on May 27, 2004.

(51) Int. Cl. A61K 31/50; A61K 31/135

(52) U.S. Cl. 514/247; 514/653

(57) ABSTRACT

This invention relates to combinations of atomoxetine, and a 5HT1_{A} receptor agonists, kits containing such combinations, pharmaceutical compositions comprising such combinations, and methods of using such combinations to treat patients suffering from ADHD, related disorders, and other central nervous system diseases or disorders.

### FIELD OF THE INVENTION

The present invention relates to pharmaceutical compositions comprising combinations of atomoxetine, a prodrug, thereof a pharmaceutically acceptable salt thereof, or a pharmaceutically acceptable salt of said prodrug, and a 5HT1A receptor agonist; kits comprising such combinations; and methods of using such combinations to treat patients, including humans, suffering from attention deficit hyperactivity disorder (ADHD) or related disorder, or other central nervous system diseases or disorders. This invention also relates to additive and synergistic combinations of atomoxetine, a prodrug thereof, a pharmaceutically acceptable salt thereof, or a pharmaceutically acceptable salt of said prodrug and a 5HT1_{A} receptor agonist, which additive and synergistic combinations are useful in treating patients, including humans, suffering from ADHD or related disorder, or other central nervous system diseases or disorders.

### BACKGROUND OF THE INVENTION

Attention deficit hyperactivity disorder (ADHD) has an estimated incidence in school age children of 3-5%, and is characterized by the core symptoms of hyperactivity, impulsivity, and/or inattention. The attentional symptoms of ADHD can be successfully treated with psychomotor stimulants such as methylphenidate (Ritalin). Clonidine, an α₂-adrenoceptor agonist, treats the aggressive and oppositional symptoms. There is a potential for significant side effects with both methylphenidate and clonidine, making it important to identify other drugs that have similar or better efficacy with reduced side effects and abuse liability.

ADHD is one of the most common childhood psychiatric disorders and appears to be a common, often underrecognized, psychiatric disease in adults as well (T. Spencer, et al., J Clin Psychiatry, 1998, 59(Suppl. 7), 759-768). This disorder, which begins in childhood, may be followed by a lifelong expression of symptoms (e.g., inattention and/or impulsivity) (J B. Schweitzer, et al., Med Clin North Am, May 2001, 85:3, 757-777). ADHD may change its manifestations as it develops from preschool through adult life (D P. Cantwell, J Am Acad Child Adolesc Psychiatry, August 1996, 35(8), 978-987; J. Elia, et al. N Eng J Med, March 1999, 340(10), 780-788; EE. Nolan, et al., J Am Acad Child Adolesc Psychaitry, February 2001, 40(2), 241-249).

The diagnosis of ADHD is based on clinical evaluation (M. Dulcan, et al. M, J Am Acad Child Adolesc Psychaitry, October 1997, 36(10 Suppl), 85S-121S; National Institutes of Health, 1998). “The essential feature of ADHD is a persistent pattern of inattention and/or hyperactivity-impulsivity that is more frequent and severe than is typically observed in individuals at a comparative level of development” (Diagnostic and Statistical Manual of Mental Disorders (DSM-IV), American Psychiatric Association, Washington, D.C., 1994). In order to be diagnosed with ADHD, patients must demonstrate symptoms of ADHD that cause impairment before the age of seven years, and symptoms must have been ongoing for longer than six months in at least two settings (e.g., school [or work] and home). (See DSM-IV).

Several NRI compounds are known. Atomoxetine, an NRI, is now commercially available (Strattera®, Eli Lilly) and is beginning to be used extensively for the clinical treatment of ADHD in both children and adults. Atomoxetine represents a non-stimulant treatment for ADHD. The number of treated ADHD patients is expected to increase as a result of the introduction of atomoxetine and enhanced educational initiatives. Accordingly, there is an ongoing need for ADHD treatments that provide more efficacy than those treatments currently available.

The present invention is directed to compositions which reduce or overcome these disadvantages. More particularly, this invention provides novel pharmaceutical combinations of atomoxetine and 5HT1_{A} receptor agonists, provided that 5HT1_{A} receptor agonist is not buspirone for the treatment of ADHD and symptoms.

### SUMMARY OF THE INVENTION

The present invention is directed to pharmaceutical compositions, therapeutic methods of treatment, and kits which employ atomoxetine together with a 5HT1_{A} receptor agonist provided that the 5HT1_{A} receptor agonist is not buspirone.

According to the invention, these pharmaceutical combinations can provide synergistic or additive effects in treating ADHD, related conditions, or other central nervous system diseases or disorders.

Thus, according to one aspect, the present invention provides a combination of atomoxetine and a 5HT1_{A} receptor agonist provided that the 5HT1_{A} receptor agonist is not buspirone. A further feature of the present invention is a method of reducing the amount of atomoxetine required treat ADHD which comprises to treating a patient with a therapeutically effective amount of a drug combination according to the present invention.

Examples of 5HT1_{A} receptor agonists that can be used in the pharmaceutical compositions of this invention include, but are not limited to: (a) sunepitron, and other bisazabicyclic compounds disclosed in U.S. Pat. No. 5,122,525 and their pharmaceutically acceptable salts, (b) gepirone; (c) ipsapirone; (d) S15535; (e) adatanserin; (f) tandospirone; (g) ipsapirone; and (h) flesinoxan.

This invention also relates to a method of treating a disorder or condition selected from the group consisting of norepinephrine dysfunction, single episodic or recurrent major depressive disorders, dysthymic disorders, depressive neurosis and neurotic depression, melancholic depression including anorexia, weight loss, insomnia, early morning waking or psychomotor retardation; atypical depression (or reactive depression) including increased appetite, hypersomnia, psychomotor agitation or irritability, seasonal affective disorder and pediatric depression; bipolar disorders or manic depression, for example, bipolar I disorder, bipolar II disorder and cyclothymic disorder; conduct disorder; attention deficit hyperactivity disorder (ADHD); disruptive behavior disorder; behavioral disturbances associated with mental retardation, autistic disorder, and conduct disorder; anxiety disorders such as panic disorder with or without agoraphobia, agoraphobia without history of panic disorder, specific phobias, for example, specific animal phobias, social anxiety, social phobia (including social anxiety disorder), obsessive-compulsive disorder and related spectrum disorders, stress disorders including post-traumatic stress disorder, acute stress disorder and chronic stress disorder, and generalized anxiety disorders; borderline personality disorder; schizophrenia and other psychotic disorders, for example, schizophreniform disorders, schizoaffective disorders, delusional disorders, brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia; delirium, dementia, and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, and other dementias, for example, due to HIV disease, head trauma, Parkinson's disease, Huntington's disease, Pick's disease, Creutzfeldt-Jakob disease, or due to multiple etiologies; movement disorders such as akinesias, dyskinesias, including familial paroxysmal dyskinesias, spasticities, Tourette's syndrome, Scott syndrome, PALSYS and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour; addictive disorders and withdrawal syndrome, chemical dependencies and addictions (e.g., dependencies on, or addictions to, alcohol, heroin, cocaine, benzodiazepines, psychoactive substances, nicotine, or phenobarbitol) and behavioral addictions such as an addiction to gambling; ocular disorders such as glaucoma and ischemic retinopathy addictive disorders (including those due to alcohol, nicotine, and other psychoactive substances) and withdrawal syndrome, adjustment disorders (including depressed mood, anxiety, mixed anxiety and depressed mood, disturbance of conduct, and mixed disturbance of conduct and mood); age-associated learning and mental disorders (including Alzheimer's disease); anorexia nervosa; apathy; attention-deficit (or other cognitive) disorders due to general medical conditions including attention-deficit disorder (ADD) and attention-deficit hyperactivity disorder (ADHD) and it's recognized sub-types; bulimia nervosa; chronic fatigue syndrome; pain; chronic pain; cyclothymic disorder; depression (including adolescent depression and minor depression); fibromyalgia and other somatoform disorders (including somatization disorder; conversion disorder; pain disorder; hypochondriasis; body dysmorphic disorder; undifferentiated somatoform disorder; and somatoform NOS); incontinence (i.e.; stress incontinence; genuine stress incontinence; and mixed incontinence); urinary disorders; premature ejaculation; inhalation disorders; intoxication disorders (alcohol addiction); mania; migraine headaches; obesity (i.e.; reducing the weight of obese or overweight patients); oppositional defiant disorder; peripheral neuropathy; diabetic neuropathy; post-herpetic neuralgic; premenstrual dysphoric disorder (i.e.; premenstrual syndrome and late luteal phase dysphoric disorder); sleep disorders (such as narcolepsy, insomnia and enuresis); specific developmental disorders; selective serotonin reuptake inhibition (SSRI) “poop out” syndrome (i.e.; wherein a patient who fails to maintain a satisfactory response to SSRI therapy after an initial period of satisfactory response); and TIC disorders (e.g.; Tourette's Disease) in a mammal, including a human, comprising administering to a mammal in need of such treatment an amount of a atomoxetine, or a pharmaceutically acceptable salt thereof, and a 5HT1_{A} receptor agonist that is effective in treating such disorder or condition provided that the 5HT1_{A} receptor agonist is not buspirone.

This invention also relates to a pharmaceutical composition for treating a disorder or condition selected from norepinephrine dysfunction, single episodic or recurrent major depressive disorders, dysthymic disorders, depressive neurosis and neurotic depression, melancholic depression including anorexia, weight loss, insomnia, early morning waking or psychomotor retardation; atypical depression (or reactive depression) including increased appetite, hypersomnia, psychomotor agitation or irritability, seasonal affective disorder and pediatric depression; bipolar disorders or manic depression, for example, bipolar I disorder, bipolar II disorder and cyclothymic disorder; conduct disorder; attention deficit hyperactivity disorder (ADHD); disruptive behavior disorder; behavioral disturbances associated with mental retardation, autistic disorder, and conduct disorder; anxiety disorders such as panic disorder with or without agoraphobia, agoraphobia without history of panic disorder, specific phobias, for example, specific animal phobias, social anxiety, social phobia (including social anxiety disorder), obsessive-compulsive disorder and related spectrum disorders, stress disorders including post-traumatic stress disorder, acute stress disorder and chronic stress disorder, and generalized anxiety disorders; borderline personality disorder; schizophrenia and other psychotic disorders, for example, schizophreniform disorders, schizoaffective disorders, delusional disorders, brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia; delirium, dementia, and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, and other dementias, for example, due to HIV disease, head trauma, Parkinson's disease, Huntington's disease, Pick's disease, Creutzfeldt-Jakob disease, or due to multiple etiologies; movement disorders such as akinesias, dyskinesias, including familial paroxysmal dyskinesias, spasticities, Tourette's syndrome, Scott syndrome, PALSYS and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour; addictive disorders and withdrawal syndrome, chemical dependencies and addictions (e.g., dependencies on, or addictions to, alcohol, heroin, cocaine, benzodiazepines, psychoactive substances, nicotine, or phenobarbitol) and behavioral addictions such as an addiction to gambling; ocular disorders such as glaucoma and ischemic retinopathy addictive disorders (including those due to alcohol, nicotine, and other psychoactive substances) and withdrawal syndrome, adjustment disorders (including depressed mood, anxiety, mixed anxiety and depressed mood, disturbance of conduct, and mixed disturbance of conduct and mood); age-associated learning and mental disorders (including Alzheimer's disease); anorexia nervosa; apathy; attention-deficit (or other cognitive) disorders due to general medical conditions including attention-deficit disorder (ADD) and attention-deficit hyperactivity disorder (ADHD) and it's recognized sub-types; bulimia nervosa; chronic fatigue syndrome; pain; chronic pain; cyclothymic disorder; depression (including adolescent depression and minor depression); fibromyalgia and other somatoform disorders (including somatization disorder; conversion disorder; pain disorder; hypochondriasis; body dysmorphic disorder; undifferentiated somatoform disorder; and somatoform NOS); incontinence (i.e.; stress incontinence; genuine stress incontinence; and mixed incontinence); urinary disorders; premature ejaculation; inhalation disorders; intoxication disorders (alcohol addiction); mania; migraine headaches; obesity (i.e.; reducing the weight of obese or overweight patients); oppositional defiant disorder; peripheral neuropathy; diabetic neuropathy; post-herpetic neuralgic; premenstrual dysphoric disorder (i.e.; premenstrual syndrome and late luteal phase dysphoric disorder); sleep disorders (such as narcolepsy, insomnia and enuresis); specific developmental disorders; selective serotonin reuptake inhibition (SSRI) “poop out” syndrome (i.e.; wherein a patient who fails to maintain a satisfactory response to SSRI therapy after an initial period of satisfactory response); and TIC disorders (e.g.; Tourette's Disease) in a mammal in need of such treatment, including a human, comprising an amount of a atomoxetine, or a pharmaceutically acceptable salt thereof, and a 5HT1_{A} receptor agonist that is effective in treating such disorder or condition provided that the 5HT1_{A} receptor agonist is not buspirone.

It is also a feature of this invention that the use of such drug combinations will enhance the effect of atomoxetine to be used and therefore allow reduced quantities of the atomoxetine to be used and, therefore allow better management of drug-related toxicity and side effects.

The invention offers advantages over previous methods for treating ADHD. The method of treatment of the present invention will enhance the effect of atomoxetine used and therefore permit reduced quantities of the atomoxetine to be used and, therefore permit improved management of drug-related toxicity and side effects. Other features and advantages of the invention will be apparent from the following detailed description and from the claims.

### DETAILED DESCRIPTION OF THE INVENTION

The present invention is directed to a pharmaceutical composition for treatment of ADHD in a mammal, including a human, comprising (a) an amount of atomoxetine, a prodrug thereof, or a pharmaceutically acceptable salt thereof, or a pharmaceutically acceptable salt of said prodrug; and (b) an amount of a 5HT1_{A} receptor agonist, provided that the 5HT1_{A} receptor agonist is not buspirone, and a pharmaceutically acceptable carrier, wherein the amounts (a) and (b) are together effective in treating ADHD or other central nervous system diseases or disorders.

The present invention is further directed to a method for treating ADHD or other central nervous system diseases or disorders in a mammal, including a human, which method comprises administering (a) an amount of atomoxetine; and (b) an amount of a 5HT1_{A} receptor agonist, with the exception of buspirone, to said mammal, wherein the amounts (a) and (b) are together effective in treating said ADHD or other central nervous system diseases or disorders.

This invention is also directed to kits for achieving a therapeutic effect in a mammal, including a human, comprising an amount of atomoxetine, a prodrug thereof or a pharmaceutically acceptable salt of atomoxetine or said prodrug and a pharmaceutically acceptable vehicle, carrier or diluent in a first unit dosage form; and an amount of a 5HT1_{A} receptor agonist provided that the 5HT1_{A} receptor agonist is not buspirone, and a pharmaceutically acceptable vehicle, carrier or diluent in a second unit dosage form, and a container.

For use in medicine, pharmaceutically acceptable salts may be useful in the preparation of the compounds according to the invention. Suitable pharmaceutically acceptable salts of the compounds of this invention include acid addition salts which may, for example, be formed by mixing a solution of the compound according to the invention with a solution of a pharmaceutically acceptable acid such as hydrochloric acid, sulfuric acid, methanesulphonic acid, fumaric acid, maleic acid, succinic acid, acetic acid, benzoic acid, oxalic acid, citric acid, tartaric acid, carbonic acid or phosphoric acid. Furthermore, where the compounds carry an acidic moiety, suitable pharmaceutically acceptable salts thereof may include alkali metal salts, e.g. sodium or potassium salts; alkaline earth metal salts, e.g. calcium or magnesium salts; and salts formed with suitable organic ligands, e.g. quaternary ammonium salts.

The expression “pharmaceutically acceptable salts” includes both pharmaceutically acceptable acid addition salts and pharmaceutically acceptable cationic salts. The expression “pharmaceutically-acceptable cationic salts” is intended to define but is not limited to such salts as the alkali metal salts, (e.g., sodium and potassium), alkaline earth metal salts (e.g., calcium and magnesium), aluminum salts, ammonium salts, and salts with organic amines such as benzathine (N,N′-dibenzylethylenediamine), choline, diethanolamine, ethylenediamine, meglumine (N-methylglucamine), benethamine (N-benzylphenethylamine), diethylamine, piperazine, tromethamine (2-amino-2-hydroxymethyl-1,3-propanediol) and procaine. The expression “pharmaceutically-acceptable acid addition salts” is intended to define but is not limited to such salts as the hydrochloride, hydrobromide, sulfate, hydrogen sulfate, phosphate, hydrogen phosphate, dihydrogenphosphate, acetate, succinate, citrate, methanesulfonate (mesylate) and p-toluenesulfonate (tosylate) salts.

The pharmaceutically-acceptable cationic salts of 5HT1_{A} receptor agonists or atomoxetine containing free carboxylic acids can be readily prepared by reacting the free acid form of the 5HT1_{A} receptor agonist with an appropriate base, usually one equivalent, in a co-solvent. Typical bases are sodium hydroxide, sodium methoxide, sodium ethoxide, sodium hydride, potassium methoxide, magnesium hydroxide, calcium hydroxide, benzathine, choline, diethanolamine, piperazine and tromethamine. The salt is isolated by concentration to dryness or by addition of a non-solvent. In many cases, salts are preferably prepared by mixing a solution of the acid with a solution of a different salt of the cation (e.g., sodium or potassium ethylhexanoate, magnesium oleate), employing a solvent (e.g., ethyl acetate) from which the desired cationic salt precipitates, or can be otherwise isolated by concentration and/or addition of a non-solvent.

The pharmaceutically acceptable acid addition salts of 5HT1_{A} receptor agonists or atomoxetine containing free amine groups can be readily prepared by reacting the free base form of the 5HT1_{A} receptor agonist with the appropriate acid. When the salt is of a monobasic acid (e.g., the hydrochloride, the hydrobromide, the p-toluenesulfonate, the acetate), the hydrogen form of a dibasic acid (e.g., the hydrogen sulfate, the succinate) or the dihydrogen form of a tribasic acid (e.g., the dihydrogen phosphate, the citrate), at least one molar equivalent and usually a molar excess of the acid is employed. However, when such salts as the sulfate, the hemisuccinate, the hydrogen phosphate or the phosphate are desired, the appropriate and exact chemical equivalents of acid will generally be used. The free base and the acid are usually combined in a co-solvent from which the desired salt precipitates, or can be otherwise isolated by concentration and/or addition of a non-solvent.

For the purposes of this specification, ADHD is defined in accordance with the DSM-IV criteria.

The expression “prodrug” refers to compounds that are drug precursors which, following administration, release the drug in vivo via a chemical or physiological process (e.g., a prodrug on being brought to the physiological pH or through enzyme action is converted to the desired drug form).

The present invention includes within its scope the use of prodrugs of atomoxetine, and of 5HT1_{A} receptor agonists. In general, such prodrugs will be functional derivatives of these compounds which are readily convertible in vivo. Conventional procedures for the selection and preparation of suitable prodrug derivatives are described, for example, in Design of Prodrugs, ed. H. Bundgaard, Elsevier, 1985 and can be achieved using methods well known to those skilled in the art. All such prodrugs are within the scope of the combinations, pharmaceutical compositions, methods and kits of this invention.

The chemist of ordinary skill in the art will also recognize that certain compounds within the scope of this invention can exist in zwifterionic form, i.e., that certain compounds contain an amine portion and a carboxylic acid portion, which, depending upon the pH of the solution, may exist as a free amine and a free carboxylic acid or as a zwitterion in which the amine is protonated to form an ammonium ion and the carboxylic acid is deprotonated to form a carboxylate ion. Use of such zwitterions are included in this invention.

The chemist of ordinary skill in the art will also recognize that some of the compounds of the pharmaceutical combinations contemplated by the present invention can exist in different stereoisomers. Specific stereoisomers may exhibit an ability to treat mental disorders with a more favorable efficacy or safety profile. The present invention includes use of all possible stereoisomers and geometric isomers of the active ingredients of each pharmaceutical combination, and includes not only racemic compounds but also optical isomers as well. In situations where tautomers, i.e. two isomers which are in rapid equilibrium with each other, are possible, the present invention is intended to include use of all tautomeric forms.

The combinations of the present invention can be administered in a standard manner for the treatment of ADHD or other central nervous system diseases or disorders such as orally, parenterally, transmucosally (e.g., sublingually or via buccal administration), topically, transdermally, rectally, via inhalation (e.g., nasal or deep lung inhalation). Parenteral administration includes, but is not limited to intravenous, intraarterial, intraperitoneal, subcutaneous, intramuscular, intrathecal, and intraarticular, or via a high pressure technique, like Powderject.™

For buccal administration, the composition can be in the form of tablets or lozenges formulated in conventional manner. For example, tablets and capsules for oral administration can contain conventional excipients such as binding agents (for example, syrup, acacia, gelatin, sorbitol, tragacanth, mucilage of starch or polyvinylpyrrolidone), fillers (for example, lactose, sugar, microcrystalline cellulose, maize-starch, calcium phosphate or sorbitol), lubricants (for example, magnesium stearate, stearic acid, talc, polyethylene glycol or silica), disintegrants (for example, potato starch or sodium starch glycollate), or wetting agents (for example, sodium lauryl sulfate). The tablets can be coated according to methods well known in the art.

Such preparations can also be formulated as suppositories for rectal administration, e.g., containing conventional suppository bases, such as cocoa butter or other glycerides. Compositions for inhalation typically can be provided in the form of a solution, suspension, or emulsion that can be administered as a dry powder or in the form of an aerosol using a conventional propellant, such as dichlorodifluoromethane or trichlorofluoromethane. Typical topical and transdermal formulations comprise conventional aqueous or nonaqueous vehicles, such as eye drops, creams, ointments, lotions, and pastes, or are in the form of a medicated plaster, patch, or membrane.

Additionally, compositions of the present invention can be formulated for parenteral administration by injection or continuous infusion. Formulations for injection can be in the form of suspensions, solutions, or emulsions in oily or aqueous vehicles, and can contain formulation agents, such as suspending, stabilizing, and/or dispersing agents. Alternatively, the active ingredient can be in powder form for constitution with a suitable vehicle (e.g., sterile, pyrogen-free water) before use.

A composition in accordance with the present invention also can be formulated as a depot preparation. Such long acting formulations can be administered by implantation (for example, subcutaneously or intramuscularly) or by intramuscular injection. Accordingly, the compounds of the invention can be formulated with suitable polymeric or hydrophobic materials (e.g., an emulsion in an acceptable oil), ion exchange resins, or as sparingly soluble derivatives (e.g., a sparingly soluble salt).

Solubilized forms of atomoxetine, pharmaceutically acceptable salts there, or prodrugs thereof, or pharmaceutically acceptable salts of prodrugs thereof, associated with (or at levels even greater than) immediate release can be fabricated into depot formulations. For example, a pharmaceutical kit comprising atomoxetine, atomoxetine salts or prodrugs thereof, or pharmaceutically acceptable salts of atomoxetine prodrugs, which can be solubilized or unsolubilized; and a constituting liquid vehicle comprised of a viscosity agent with the proviso that when the atomoxetine compound is unsolubilized, the aqueous liquid further comprises a solubilizer.

For oral administration a pharmaceutical composition can take the form of solutions, suspensions, tablets, pills, capsules, powders, and the like. Tablets containing various excipients such as sodium citrate, calcium carbonate and calcium phosphate are employed along with various disintegrants such as starch and preferably potato or tapioca starch and certain complex silicates, together with binding agents such as polyvinylpyrrolidone, sucrose, gelatin and acacia. Additionally, lubricating agents such as magnesium stearate, sodium lauryl sulfate and talc are often very useful for tabletting purposes. Solid compositions of a similar type are also employed as fillers in soft and hard-filled gelatin capsules; preferred materials in this connection also include lactose or milk sugar as well as high molecular weight polyethylene glycols.

Alternatively, the compounds of the present invention can be incorporated into oral liquid preparations such as aqueous or oily suspensions, solutions, emulsions, syrups, or elixirs, for example. Moreover, formulations containing these compounds can be presented as a dry product for constitution with water or other suitable vehicle before use. Such liquid preparations can contain conventional additives, such as suspending agents, such as sorbitol syrup, synthetic and natural gums such as tragacanth, acacia, alginate, dextran, sodium carboxymethylcellulose, methylcellulose, polyvinyl-pyrrolidone or gelatin, glucose/sugar syrup, gelatin, hydroxyethylcellulose, hydroxypropylmethylcellulose, aluminum stearate gel, emulsifying agents, such as lecithin, sorbitan monooleate, or acacia; nonaqueous vehicles (which can include edible oils), such as almond oil, fractionated coconut oil, oily esters, propylene glycol, and ethyl alcohol; and preservatives, such as methyl or propyl p-hydroxybenzoate and sorbic acid. The liquid forms in which the compositions of the present invention may be incorporated for administration orally or by injection include aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles.

When aqueous suspensions and/or elixirs are desired for oral administration, the compounds of this invention can be combined with various sweetening agents, flavoring agents, coloring agents, emulsifying agents and/or suspending agents, as well as such diluents as water, ethanol, propylene glycol, glycerin and various like combinations thereof. Suitable dispersing or suspending agents for aqueous suspensions include synthetic and natural gums such as tragacanth, acacia, alginate, dextran, sodium carboxymethylcellulose, methylcellulose, polyvinyl-pyrrolidone or gelatin.

The combinations of this invention can also be administered in a controlled release formulation such as a slow release or a fast release formulation. Such controlled release formulations of the combinations of this invention may be prepared using methods well known to those skilled in the art. The method of administration will be determined, by the attendant physician or other person skilled in the art after an evaluation of the patient's condition and requirements.

By the term “controlled release” is meant release of the active substance from the dosage form is modified to occur at a slower rate than that from an immediate release product, such as a conventional swallow tablet or capsule.

By the term “immediate release” is meant a pharmaceutical composition in which one of more active ingredients therein demonstrates at least about 80-100% (w/v) dissolution, preferably between from about 90% (w/v) to about 95% (w/v) within about 15 to 20 minutes as determined by a standard dissolution test. Suitable standard dissolution tests are known in the field.

The pharmaceutical compositions of the present invention can consist of a combination of immediate release and controlled release characteristics. Such compositions can take the form of combinations of the active ingredients that range in size from nanoparticles to microparticles or in the form of a plurality of pellets with different release rates. The tablet or capsule composition of the present invention can contain an atypical antipsychotic in sustained or controlled release form and, a second therapeutic agent in an immediate release form. Alternatively, the atypical antipsychotic can be in immediate release form and the second therapeutic agent can be in sustained or controlled release form.

The combinations of this invention can also be administered in parenteral form. For parenteral administration, solutions in sesame or peanut oil or in aqueous propylene glycol can be employed, as well as sterile aqueous solutions of the corresponding water-soluble salts. Such aqueous solutions can be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well-known to those skilled in the art.

Methods of preparing various pharmaceutical compositions with a certain amount of active ingredient are known, or will be apparent in light of this disclosure, to those skilled in this art. For examples, methods of preparing pellets are described in Remington: The Science and Practice of Pharmacy, Mack Publishing Company, Easton, Pa., 19th Edition (1995). Prolonged release pellets are prepared by either coating immediate release pellets or via matrix systems. Coating may be carried out, for example, in coating pans or in fluid bed coater-driers. Extrusion and subsequent spheronization is a long-known method for the preparation of pharmaceutical pellets (J. W. Conine et al., Drug & Cosmetic Ind. 106: 38-41; (1970)). However, other methods such as pelletization may be utilized. Particles may be agglomerated to form spherical granules or pellets, in a high speed mixer granulator, or rotary fluid bed agglomerator. These methods are described by K. W. Olson and A. M. Mehta, Int. J. Pharm. Tech & Prod. Mfr., 6: 18-24; (1985). Pellets may be also prepared by extrusion of wet masses or melts followed by spheronisation, for example as described in C. Vervaet, L. Baert & J. P. Remon, Int. J. Pharm. 116: 131-146; (1995). Excipients used are typically those with plastic qualities such as microcrystalline cellulose, but also mannitol. Small quantities of a polymeric binder are generally added. Surfactants such as sodium dodecyl sulphate may also be incorporated to give easier extrusion.

Pharmaceutical compositions according to the invention can contain 0.1%-95% of the therapeutic agents of this invention, preferably 1%-70%. In any event, the composition or formulation to be administered will contain a quantity of therapeutic agent(s) according to the invention in an amount effective to treat the condition or disease of the subject being treated.

The two different active ingredients of the compositions of this invention can be co-administered simultaneously or sequentially in any order, or as a single pharmaceutical composition comprising, for example, atomoxetine and a 5HT1_{A} receptor agonist as described above.

Since the present invention has an aspect that relates to the treatment of the disease/conditions described herein with a combination of active ingredients, which can be administered separately, the invention also relates to combining separate pharmaceutical compositions in kit form. The kit comprises two separate pharmaceutical compositions: atomoxetine and a 5HT1_{A} receptor agonist, a prodrug thereof or a pharmaceutically acceptable salt of said 5HT1_{A} agonist or prodrug. The kit includes a container for containing the separate compositions such as a divided bottle or a divided foil packet. Typically the kit includes directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, . . . etc. . . . Second Week, Monday, Tuesday, . . . ” etc. Other variations of memory aids will be readily apparent to the skilled practitioner. A “daily dose” can be a single tablet or capsule or several pills or capsules to be taken on a given day. Also, a daily dose of the atomoxetine can consist of one tablet or capsule while a daily dose of the 5HT1_{A} receptor agonist can consist of several tablets or capsules or vice versa. The memory aid should reflect this.

In another specific embodiment of the invention, a dispenser designed to dispense the daily doses one at a time in the order of their intended use is provided. Preferably, the dispenser is equipped with a memory-aid, so as to further facilitate compliance with the regimen. An example of such a memory-aid is a mechanical counter, which indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

It will be understood that while the use of a 5HT1_{A} receptor agonist as a second component compound is preferred, combinations of two or more of these agents can be used as a second component if necessary or desired.

It is preferred that the total amount ranges from about 0.0001 to about 1000 mg/kg per day, more preferably from about 0.01 to about 100 mg/kg per day and most preferably from about 0.1 to about 60 mg/kg per day.

Pharmaceutical compositions of use in the present invention will contain one or both active compound(s) in association with a pharmaceutically acceptable carrier. Preferably these compositions are in unit dosage forms such as tablets, pills, capsules, powders, granules, sterile parenteral solutions or suspensions, metered aerosol or liquid sprays, drops, ampules, auto-injector devices or suppositories; for oral, parenteral, intranasal, sublingual or rectal administration, or for administration by inhalation or insufflation. For preparing solid compositions such as tablets, the principal active ingredients are mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof.

When referring to these preformulation compositions as “homogeneous”, it is meant that the active ingredients are dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 2000 mg of each of the active ingredients of the present invention. Typical unit dosage forms contain from 1 to 300 mg, for example 1, 2, 5, 10, 25, 50 or 100 mg, of the active ingredient. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

When administered in combination, either as a single or as separate pharmaceutical composition(s), the atomoxetine and the 5HT1_{A} receptor agonist are presented in a ratio which is consistent with the manifestation of the desired effect. In particular, the ratio by weight of atomoxetine to the 5HT1_{A} receptor agonist will suitably be between 0.001 to 1 and 1000 to 1, and especially between 0.01 to 1 and 100 to 1.

The pharmaceutical combinations can be administered on a regimen of up to 6 times per day, preferably 1 to 4 times per day, especially 2 times per day, and most especially once daily.

As used herein the term “mammal” includes animals of economic importance such as bovine, and porcine animals, especially those that produce meat, as well as domestic animals (e.g. cats and dogs), sports animals (e.g. horses), zoo animals, and humans, the latter being most preferred.

### EXAMPLE 1

A pharmaceutical composition is prepared by combining atomoxetine with a 5HT1_{A} receptor agonist, which is buspirone, in a pharmaceutically acceptable carrier. The composition contains respective amounts of atomoxetine and buspirone to deliver on a daily basis between about 0.1 mg to about 1000 mg atomoxetine and a therapeutically effective amount of the 5HT1_{A} receptor agonist. The composition is administered to a patient for the treatment of ADHD and/or cognitive impairment on a daily, twice daily, three times daily, or four times daily basis.

It should be understood that the invention is not limited to the particular embodiments and examples described herein, but that various changes and modifications may be made without departing from the spirit and scope of this novel concept as defined by the following claims.

1. A pharmaceutical composition for treating ADHD in a mammal comprising (a) an amount of a first therapeutic agent which is atomoxetine and (b) an amount of a second therapeutic agent which is a 5HT1_{A} receptor agonist, wherein the amounts of (a) and (b) are together effective in treating the ADHD, provided that the 5HT1_{A} receptor agonist is not buspirone.

2. The pharmaceutical composition of claim 1, wherein the second agent is selected from (a) sunepitron, (b) gepirone, (c) ipsapirone, (d) S15535, (e) adatanserin, (f) tandospirone, (g) ipsapirone, and (h) and flesinoxan, a prodrug thereof, a pharmaceutically acceptable salt thereof, or a pharmaceutically acceptable salt of said prodrug.

3. A method for treating ADHD in a subject in need thereof comprising administering to said subject
a) an amount of a first therapeutic agent which is atomoxetine; and
b) an amount of a second therapeutic agent which is a 5HT1_{A} receptor agonist;
wherein the amounts of (a) and (b) are together effective in treating said ADHD, provided that the 5HT1_{A} receptor agonist is not buspirone.

4. The method of claim 3, wherein the first therapeutic agent is atomoxetine or a pharmaceutically acceptable salt thereof, or a prodrug thereof, or a pharmaceutically acceptable salt of said prodrug.

5. The method of claim 3, wherein (a) and (b) are administered to the subject in a single pharmaceutical composition additionally comprising a pharmaceutically acceptable vehicle, carrier or diluent, by a method selected from the group consisting of oral, transmucosal, transdermal, nasal, pulmonary, buccal, parenteral, rectal, and sublingual.

6. The method of claim 5, wherein parenteral administration to the subject is selected from the group consisting of intravenous, intramuscular, subcutaneous, and intradermal.

7. A method for treating a central nervous system disease or disorder in a subject in need thereof comprising administering to said subject
a) an amount of a first therapeutic agent which is atomoxetine; and
b) an amount of a second therapeutic agent which is a 5HT1_{A} receptor agonist;
wherein the amounts of (a) and (b) are together effective in treating said central nervous system disease or disorder, provided that the 5HT1_{A} receptor agonist is not buspirone; wherein the central nervous system disease or disorder is selected from the group consisting of dysthymic disorders, seasonal affective disorder, conduct disorders including disturbance of conduct, mixed disturbance of conduct and mood, oppositional defiant disorder, and disruptive behavior disorder; adjustment disorders including depressed mood, anxiety, and mixed anxiety; attention-deficit or other cognitive disorders due to general medical conditions including attention-deficit disorder (ADD) and attention-deficit hyperactivity disorder (ADHD) and it's recognized sub-types; and movement disorders such as akinesias, dyskinesias, spasticities, and Tourette's syndrome; chronic fatigue syndrome, fibromyalgia and other somatoform disorders including somatization disorder; pain, chronic pain, peripheral neuropathy, diabetic neuropathy, post herpetic neuropathy; and premenstrual dysphoric disorder including premenstrual syndrome and late luteal phase dysphoric disorder.

8. The method of claim 7, wherein the first therapeutic agent is atomoxetine, a pharmaceutically acceptable salt of atomoxetine, a prodrug of atomoxetine or a pharmaceutically acceptable salt of said prodrug.

9. The method of claim 7, wherein (a) and (b) are administered to the subject in a single pharmaceutical composition additionally comprising a pharmaceutically acceptable vehicle, carrier or diluent, by a method selected from the group consisting of oral, transmucosal, transdermal, nasal, pulmonary, buccal, parenteral, rectal, and sublingual.

10. The method of claim 9, wherein parenteral administration to the subject is selected from the group consisting of intravenous, intramuscular, subcutaneous, and intradermal.

11. The method of claim 7, wherein said second agent is selected from (a) sunepitron, (b) gepirone, (c) ipsapirone, (d) S15535, (e) adatanserin, (f) tandospirone, (g) ipsapirone, and (h) and flesinoxan, a prodrug thereof, a pharmaceutically acceptable salt thereof, or a pharmaceutically acceptable salt of said prodrug.

12. A kit for treating a subject having ADHD or a central nervous system disease or disorder as defined in claim 7, said kit comprising
a) a package containing a unit dosage of atomoxetine, a prodrug thereof, a pharmaceutically acceptable salt of atomoxetine, or a pharmaceutically acceptable salt of said prodrug; and
b) a package containing a unit dosage of a 5HT1_{A} receptor agonist, provided that the 5HT1_{A} receptor agonist is not buspirone.

13. The kit of claim 12, wherein said atomoxetine, atomoxetine salt, atomoxetine prodrug or salt of atomoxetine prodrug is provided in more than one unit dosage, and wherein said 5HT1_{A} receptor agonist is provided in more than one unit dosage.
